(19) United States
(12) Patent Application Publication
(10) Pub. No.: US 2006/0151297 A1
(43) Pub. Date: Jul. 13, 2006

(54) Clean room guided conveyor

(76) Inventors: Adrian L. Pyke, Reading, MA (US); William T Lebo, Quincy, MA (US)

Correspondence Address: WEINGARTEN, SCHURGIN, GAGNEBIN & LEBOVICI LLP, TEN POST OFFICE SQUARE, BOSTON, MA, 02109, US

(21) Appl. No.: 10/562,755

(22) PCT Filed: Jul. 2, 2004
(86) PCT No.: PCT/US04/21561

§ 371 Date: Dec. 29, 2005

(30) Foreign Application Priority Data
Jul. 3, 2003 (US) 60484789

(51) Int. Cl. A61K 51/00

(52) U.S. Cl. 198/780; 438/800; 414/940

(57) ABSTRACT

A conveyor system and method that reduces extraneous forces in a manufacturing process is disclosed. The system and method of the invention are particularly directed to handling and processing sensitive materials such as, for example, in clean room manufacturing facilities. These facilities are used in the fields of semiconductor fabrication, hard disk drive manufacturing, biotechnology, biomedical engineering and pharmaceutical engineering. A conveyor system according to the invention includes a carrier and a drive assembly for providing relative movement thereto. A rib or groove extends along the bottom of the carrier parallel to the direction of travel. The rib and groove structures are also intended to reduce mechanical contact between the carrier and the drive assembly, including its components. A reduction of such contact limits the extent of contamination caused by particulate formation.

### CROSS REFERENCE TO RELATED APPLICATIONS

This application claims the priority of U.S. Provisional Application No. 60/484,789 filed on Jul. 3, 2003 entitled, CLEAN ROOM GUIDED CONVEYOR, the whole of which is hereby incorporated by reference herein.

### STATEMENT REGARDING FEDERALLY SPONSORED RESEARCH OR DEVELOPMENT

Not applicable.

### BACKGROUND OF THE INVENTION

In general, most manufacturing processes include automated operations to improve efficiency and overall product quality. One concern with automation is that materials or products may be mishandled or damaged. These concerns are more significant for manufacturing processes involving sensitive materials such as, for example, in the fields of semiconductor fabrication, hard disk drive manufacturing, biotechnology, biomedical engineering and pharmaceutical engineering.

These fields commonly use clean room facilities to reduce the likelihood of the process becoming contaminated. Contamination is a particular issue with semiconductor wafers. Conveyor systems are used in clean rooms to transport these wafers for processing. The systems are designed to quickly move wafer payloads without creating particles that could contaminate the wafers. It is also desired to have a system in which the external forces acting on the wafers are minimal.

To achieve these goals, a conveyor system must be stable and move in a consistent manner. The system should also not produce additional contaminants, which occurs when a surface comes into frictional mechanical contact with another surface. It is also useful to design a conveyor system which requires a minimal driving force to be moved.

The majority of existing conveyor systems impart undesired external forces to the wafer payload. These systems also have numerous surfaces that are in frictional mechanical contact with other surfaces. Moreover, there are few systems that are quick enough to meet ever increasing process demands. Another design obstacle to consider is the cost of conveyor construction materials.

### SUMMARY OF THE INVENTION

The present invention is directed to a conveyor system that can be used in a clean room facility. The invention is particularly useful for systems that handle sensitive materials. These materials are common in the fields of semiconductor fabrication, hard disk drive manufacturing, biotechnology, biomedical engineering and pharmaceutical engineering.

A conveyor system of the invention includes a carrier or vehicle propelled by a drive assembly. The carrier generally has a main body and a bottom. The bottom is designed to have a rib or groove that is parallel to the carrier's direction of travel. Preferably, the system is designed to curtail undesired external forces from acting on the carrier. This is accomplished by providing a guidance mechanism which minimizes frictional contact while controlling lateral deviation from an intended path of carrier travel. The formation of particulate matter is minimized, as is the application of sudden lateral forces to the carrier and its contents by this guidance mechanism.

The invention is also directed to a method of using the described conveyor system. The method includes actuating the system and continuously or selectively moving the carrier.

### DESCRIPTION OF THE DRAWINGS

The invention will be more fully understood by reference to the following detailed description of the invention in conjunction with the drawings, of which:

FIG. 1 is a pictorial view of a first embodiment of the conveyor system according to the invention;

FIG. 2 is a detailed view of a carrier and conveyor of the system of FIG. 1;

FIG. 3 is a section view of the conveyor system of FIG. 1;

FIG. 4 is a partial section view of the conveyor system of FIG. 1;

FIG. 5 is a pictorial view of a second embodiment of the conveyor system according to the invention;

FIG. 6 is a detailed view of a carrier and conveyor of the system of FIG. 5;

FIG. 7 is a section view of the conveyor system of FIG. 5;

FIG. 8 is a partial section view of the conveyor system of FIG. 5;

FIG. 9 is a pictorial view of a third embodiment of the conveyor system according to the invention;

FIG. 10 is a detailed view of a carrier and conveyor of the system of FIG. 9;

FIG. 11 is a section view of the conveyor system of FIG. 9;

FIG. 12 is a partial section view of the conveyor system of FIG. 9;

FIG. 13 is a detailed view of the system of FIG. 9; and

FIG. 14 is a detailed view of the system of FIGS. 1 or 5.

### DETAILED DESCRIPTION OF THE INVENTION

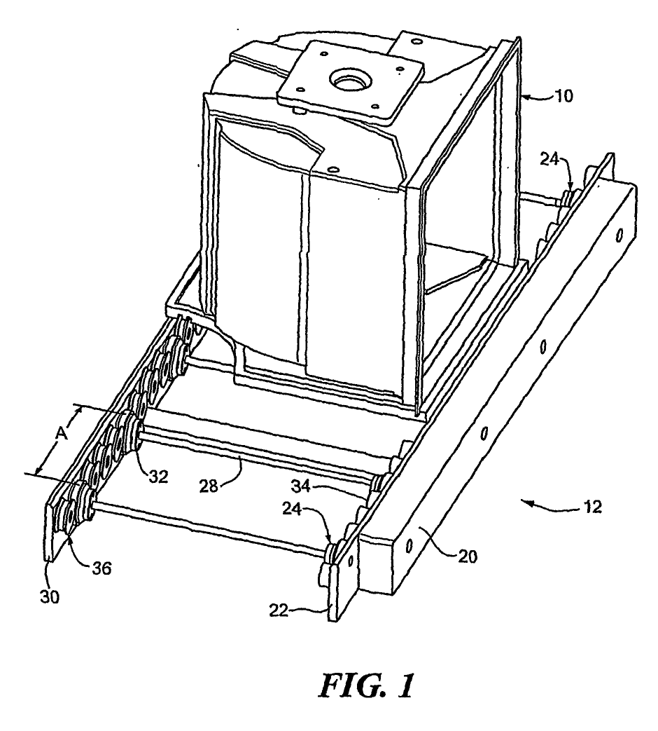

The present invention is useful for transporting sensitive materials in a clean room. In a first embodiment, the invention is directed to the conveyor system shown in FIGS. 1-4. The system includes a carrier 10 and a conveyor assembly 12. The bottom surface 14 of the carrier has a rib 16 that is parallel to the direction of travel of the carrier. This rib extends the length of the carrier bottom surface. The rib may be recessed so as to not extend beyond the bottom surface of the carrier, may be flush with the bottom surface, or may extend beyond the bottom surface. While a recessed rib may be less likely to interfere with other components of the carrier system and may experience less wear, a projecting rib may be useful for supporting the carrier on a solid surface. The rib preferably has a square or rectangular cross-section.

Alternative embodiments of the rib may include designs in which the rib does- not extend the length of the carrier 10 or those that have a plurality of discrete ribs on the bottom of the carrier. Although the preferred rib is formed integral with the carrier, other designs may include a rib that is, for example, welded or fastened to the bottom of the carrier. Furthermore, there may be several continuous ribs that extend the length of the carrier.

Preferably, the system of FIGS. 1-4 is used to transport semiconductor wafers. The wafers are retained in the carrier 10. The form of the carrier may vary depending on the type of manufacturing process in which it is used. For example, a carrier for merely transporting wafers may be different from one that suspends or supports a wafer during fabrication processes.

FIGS. 1 and 3-4 also show a drive assembly 20 associated with the conveyor assembly 12 of the system. The drive assembly 20 houses the driving motors that propel the carrier 10. The assembly also includes sensors or other process controls that actuate and control the movement of the carrier 10. Alternatively, the assembly may include hydraulic, pneumatic or electronic control systems.

Fixed to the drive assembly 20 is a drive rail 22. Coupled to the drive rail are drive wheels 24 that contact the bottom surface 14 of the carrier 10. A circumferential surface of the drive wheels is preferably provided as a resilient, durable material such as, for example, hardened rubber. The drive wheels are coupled to the drive rail and drive assembly by drive shafts 26. The drive shafts extend from the interior of the drive assembly, where they are directly or indirectly coupled to motors (not shown), through the drive rail, and into the drive wheels. The drive wheels are rotated as the drive shafts rotate. The drive shafts are preferably substantially cylindrical, although other geometries can be employed. When the drive shafts pass through the midpoint of the drive wheels, balanced rotation is achieved.

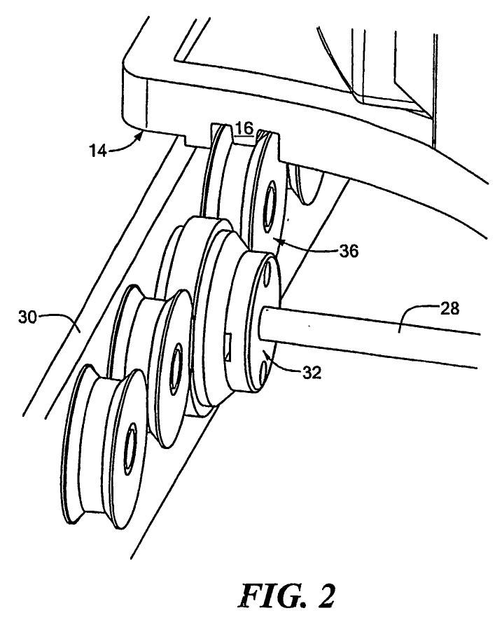

The drive shafts 26 may extend to a complimentary wheel on the other side of the conveyor assembly 12 or may be disposed with an axle 28, as illustrated. In the embodiment illustrated in FIGS. 1 and 2, the axle extends between drive wheels 24 proximate the drive rail 22 to slave wheels 32 proximate a base rail 30. The slave wheels are optionally similar to or the same as the drive wheels. The base rail is generally aligned in parallel with the drive rail. The distance between the base and drive rail will depend upon the process for which the conveyor system is used and the dimensions of the carrier.

The axle 28 may extend through the slave wheels 32 into the base rail 30, or slave shafts (not shown), similar to the drive shafts 26, may be employed. Because the drive shafts and/or axles couple the drive wheels 24 to the slave wheels, the rotation of both wheels is synchronized. This synchronized rotation allows the carrier 10 to be uniformly propelled.

Although the drive and slave wheels 24, 32 are illustrated as occupying the same respective horizontal plane, a slave shaft offset may be used when the slave wheels are not all in the same plane. In addition, other alternative embodiments may not include slave wheels such that the carrier 10 is supported and propelled by the drive wheels. The drive wheels 24 and drive assemblies 20 may also be provided on both sides of the conveyor assembly 12.

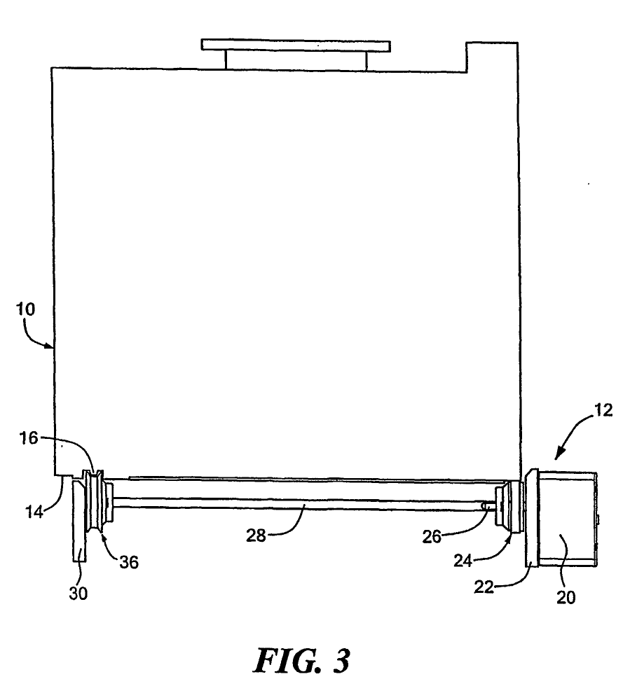

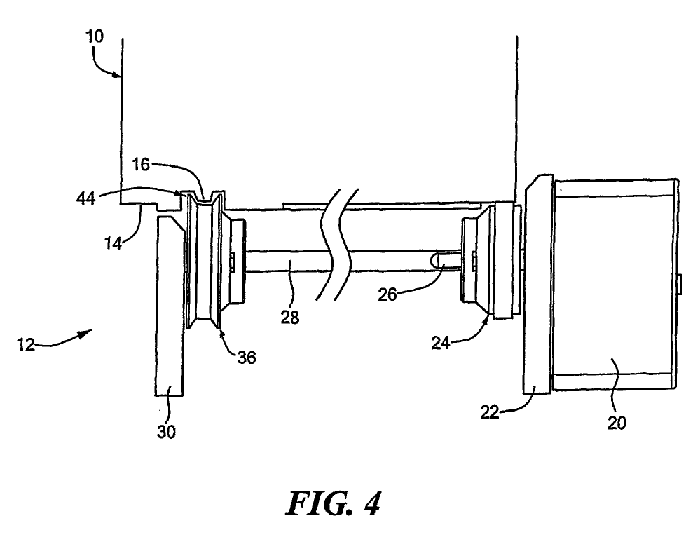

In the embodiment of FIGS. 3 and 4, guide wheels 36 take the place of slave wheels 32 and so are in mechanical communication with the drive assembly via the axle 28.

The drive wheels 24 and slave wheels 32 (FIGS. 1 and 2) or guide wheels 36 (FIGS. 3 and 4) uniformly propel the carrier 10 while also supporting the weight of the carrier. The weight of the carrier may also be supported by passive or non-driven idler wheels 34, as shown in FIG. 1. The need for these idler wheels 34, for guide wheels 36 in addition to slave wheels 32 (FIGS. 1 and 2), and other wheels or embodiments subsequently described, depends on the length of the carrier bottom surface 14 relative to the spacing of the drive wheels. When the length of the carrier bottom surface is short compared to the spacing of the drive wheels, idler wheels and passive guide wheels (FIGS. 1 and 2) are necessary to support the weight of the carrier. In contrast, when the length of the carrier bottom surface is long compared to the spacing of the drive wheels, idler wheels are not necessary, guide wheels take the place of the slave wheels (FIGS. 3 and 4), and the weight of the carrier may be supported by the drive wheels and the guide wheels coupled to the drive wheels.

FIG. 2 shows the carrier bottom surface 14, a rib 16, and one of a series of guide wheels 36 disposed directly beneath the rib 16. The purpose of the guide wheels 36 in this embodiment is to maintain the carrier 10 in the proper lateral position with respect to the conveyor assembly 12. FIG. 14 also provides a detailed view of the rib and a guide wheel. The guide wheel is provided with a central circumferential surface 38 having a constant radial distance from an axis of symmetry through the guide wheel. On either side of the central surface are edge circumferential surfaces 40 separated from the central surface by respective transitional surfaces 42. The guide wheel of FIG. 14 is shown in contact with the lower surface of the rib 16, thus assisting with bearing the weight of the carrier. In alternative embodiments, the guide wheels are disposed such that the central surface is just below the lowest extent of the rib. In either case, if the carrier begins to translate laterally with respect to the intended path of travel, a lateral edge of the rib will come into point contact with one of the transitional surfaces, thereby urging the carrier back into the proper lateral position. Due to the point contact nature of the interface between the rib and the guide wheel, opportunities for particulate generation are minimized.

In the embodiment of FIGS. 1-2, guide wheels 36 are shown interposed between slave wheels 32 on the base rail 30 side of the conveyor assembly 12. As discussed above, if the dimension A between successive driven wheels is not too great with respect to the length of the rib 16 along the carrier bottom surface 14, the guide wheels need not be in continuous contact with the lowest extent of the rib in order to serve a weight bearing function or may take the place of the slave wheel on the axle 28 and provide both driving and weight bearing functions. If, however, dimension A in FIG. 1 is greater than the rib length, or is so great that the carrier may not be stable as it passes between slave wheels, the passive guide wheels may be in continuous contact with the rib lower surface, as shown in FIGS. 1 and 2.

In FIG. 1, the drive wheels 24 and slave wheels 32 are shown to be similarly designed. These designs allow the outer circumferential surface of the drive wheels to be in flush contact with the bottom surface 14 of the carrier 10. Similarly, the outer circumferential surface of the slave wheels is in flush contact with the lower extent of the rib 16 of the carrier. Contact of the carrier by the drive and slave wheels is generally intended to straddle the carrier's center of gravity. Thus, the drive and slave wheels are preferably positioned equidistant from the carrier's center of gravity.

Different processes and applications, however, may require that the drive and slave wheels 24, 32 contact the bottom surface of the carrier 10 at different locations. Moreover, additional wheels, slideable members or a third rail may also change the location of the drive and slave wheels. The drive and slave wheels may also contact the main body of the carrier in addition to or rather than at the carrier bottom surface. It will be appreciated that a multitude of wheels and rail configurations allow the conveyor system to be used in manufacturing process having a range of dimensions and layouts such as, for example, those which have turns or an incline and decline.

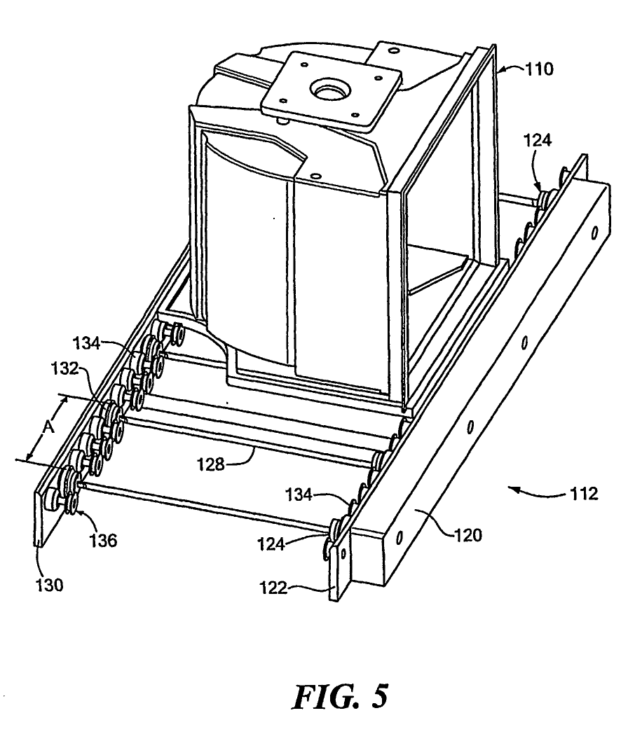

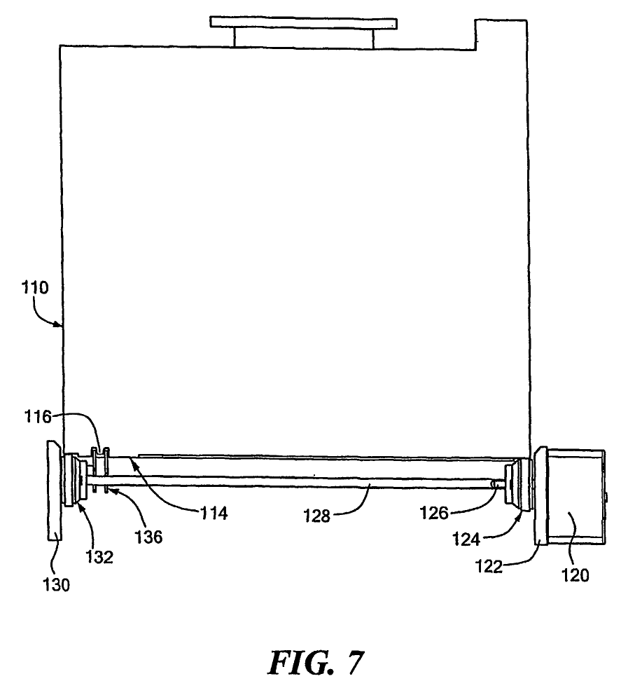

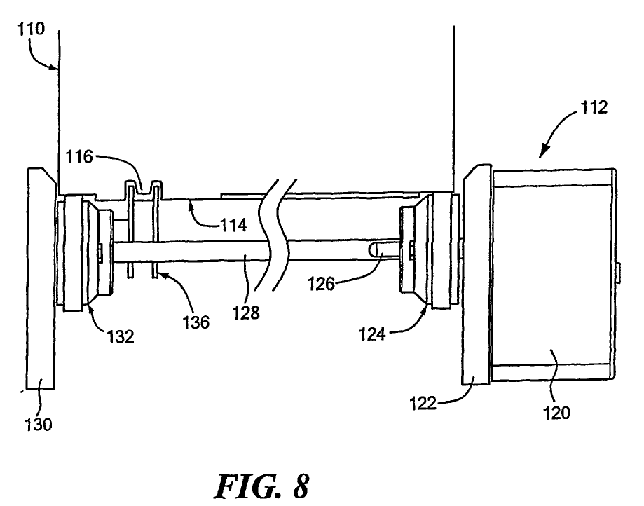

Another embodiment of the conveyor system is shown in FIGS. 5-8. This embodiment is similar to the previously described embodiments except that the rib 116, which extends along the bottom surface 114 of the carrier 110, is merely used for lateral guidance. Also, the base rail 130 (as well as the drive rail 122) is provided with idler wheels 134 interposed between the slave wheels 132. As in the previous embodiment, the number of idler wheels between slave wheels (or between drive wheels 124 on the drive rail side) depends on various factors including, but not limited to, the carrier bottom dimensions, etc. The idler wheels 134 are used to support the carrier.

In this embodiment, the drive assembly 120, drive rail 122, drive shafts 126 and axles 128 are substantially identical to that of the previously described embodiment.

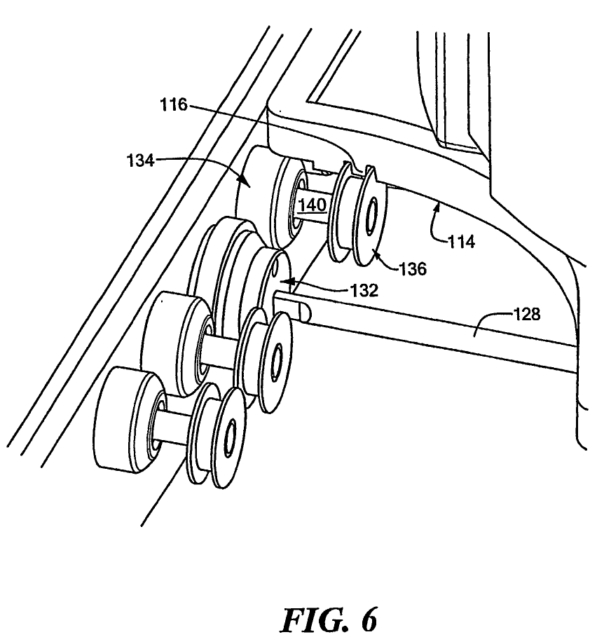

Guidance of the carrier in this embodiment is accomplished by guide wheels 136, which are similar to those in the embodiment of FIGS. 1-4, though here they are laterally offset from the slave and idler wheels 132, 134 on the base rail 130 side of the conveyor assembly 112. Since idler wheels are provided on the base rail side of the conveyor assembly in this embodiment, there is no requirement for the guide wheels to be weight bearing. Thus, as shown in FIGS. 6-8, there is a gap between the lower extent of the rib 116 and the central circumference of the guide wheel 136. While this guide wheel is shown without transitional surfaces intermediate to the edge circumferential surfaces and the central circumferential surface such transitional surfaces may also be employed as previously described.

As shown especially in FIG. 6, each of the guide wheels 136 shares an axis of rotation with a respective idler wheel 134. A shaft 140 may be fixed with respect to the base rail 130, allowing the idler and guide wheels to independently rotate thereabout.

It will also be appreciated that the various alternative designs or constructions of the previously described embodiments are applicable to that shown in FIGS. 5-8. These alternative designs or constructions include, but are not limited to, different rib positions and complimentary wheel locations.

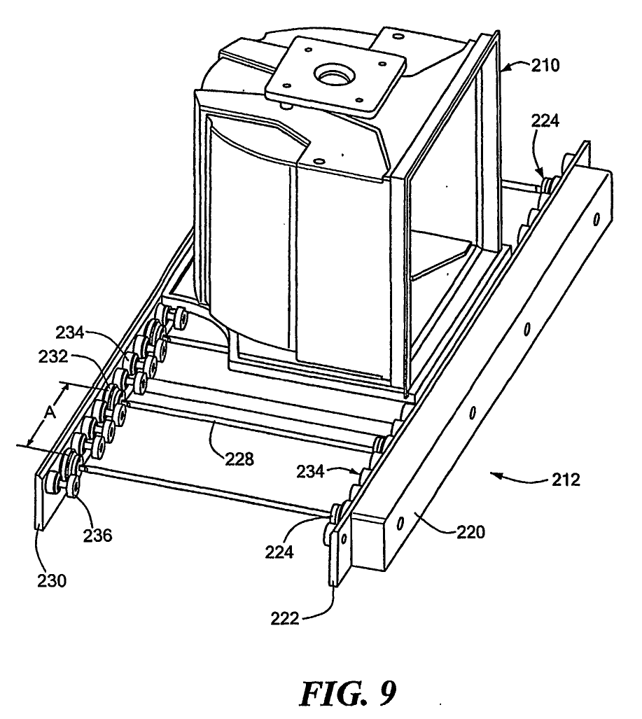

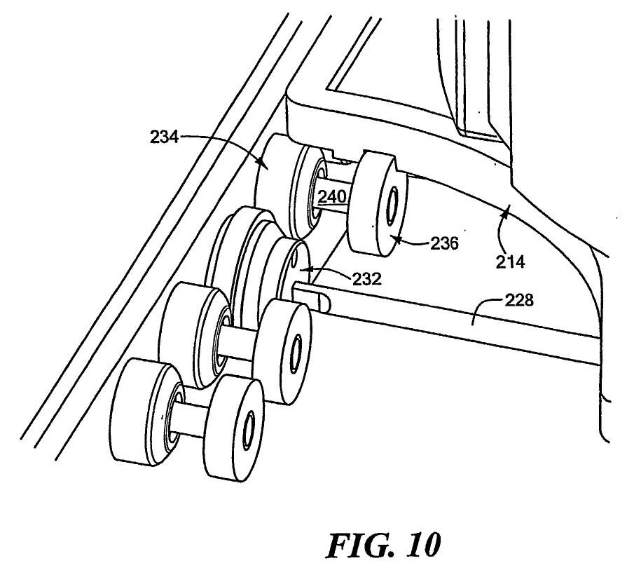

The rib structure previously described is substituted by a groove 216 in an alternative embodiment of the conveyor system illustrated in FIGS. 9-12. This grove provides lateral guidance for the carrier 210. Lateral guidance is accomplished by guide wheels 236 that travel within the groove. The guide wheels 236 in this embodiment are preferably provided with a simple outer circumference of constant diameter. Because the rib structure is replaced by a groove, any channels (e.g., channels 44 in FIG. 14) associated with the rib are not required.

Otherwise, this embodiment is similar to those embodiments previously described. For instance, as illustrated, the guide wheels 236 may be disposed on a shaft 240 similar to the offset position of the guide wheel 136 in FIG. 6. Thus, the guide wheel shares the shaft with an idler wheel 234. The idler wheel is substantially aligned with a slave wheel 232, which is shown sharing an axle 228 with a drive wheel 224 on a drive shaft 226.

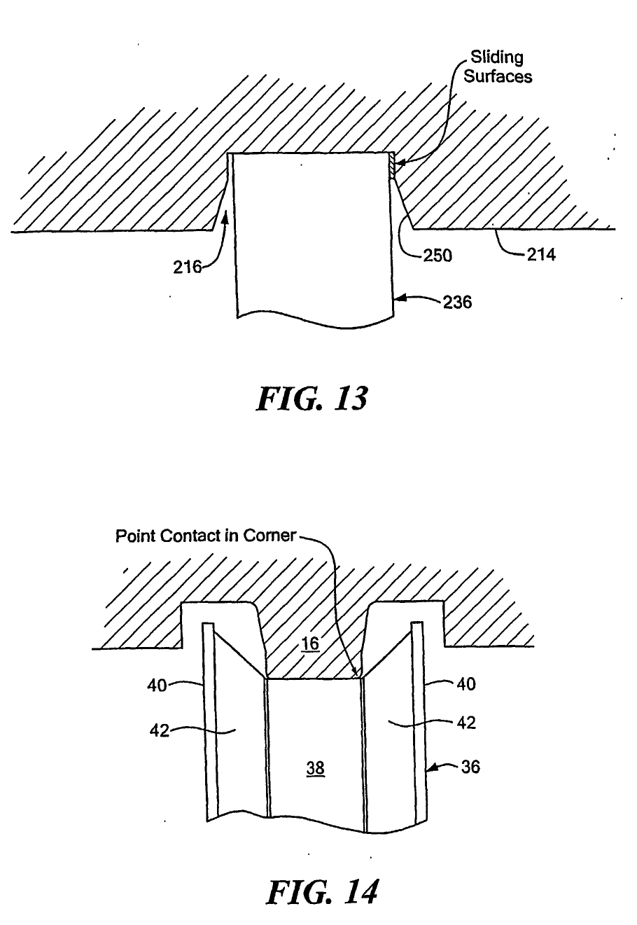

With reference to FIG. 13, the groove 216 is preferably formed with side walls that include at least a portion which is angled away from the groove. This minimizes the contact area between the guide wheel 236 and the carrier bottom surface 214 forming the groove, which minimizes the opportunity for particulate generation. Preferably, the groove, at its minimum width, is wider than the guide wheel 236 such that contact between the two only occurs in opposition to lateral forces applied to the carrier 210.

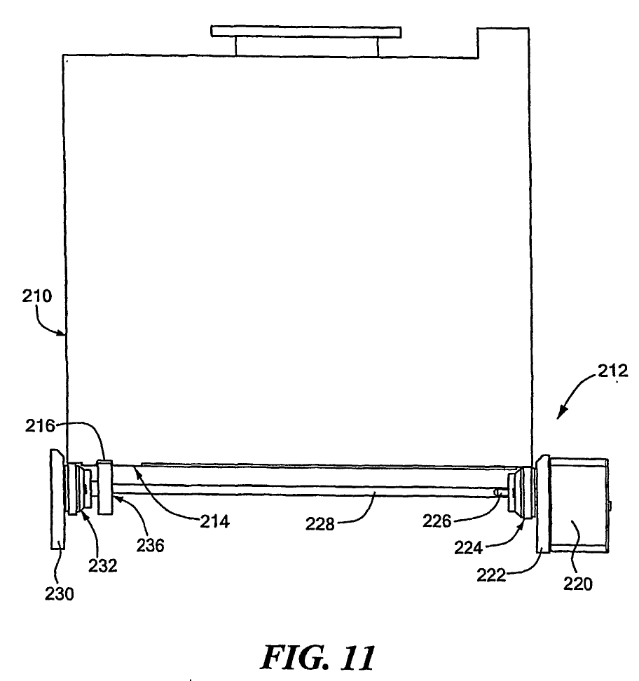

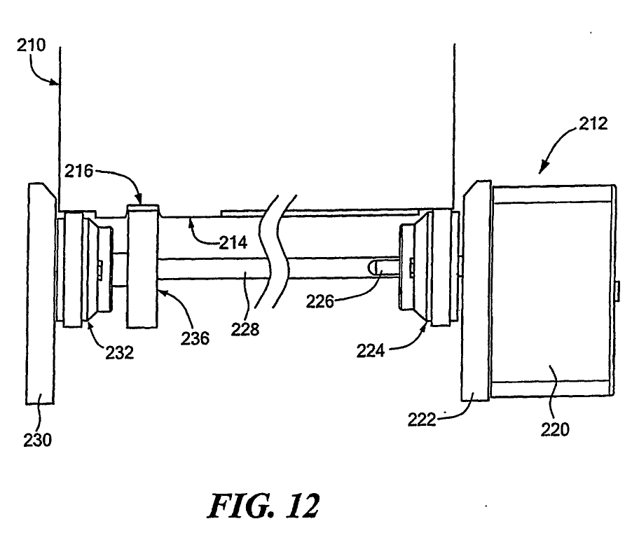

While the guide wheel 236 is shown in contact with a upper extent of the groove 216 in FIG. 13, thus illustrating a weight bearing function, the outer circumference of the guide wheel may also extend just below the upper extent of the groove 216, such as shown in FIGS. 11 and 12.

It will also be appreciated that the various alternative designs or constructions of the previously described embodiments are applicable to the embodiments shown in FIGS. 9-12. These alternative designs or constructions include, but are not limited to, different rib or wheel positions. Furthermore, these variations are applicable to any of the embodiments described herein.

It is understood in describing the conveyor system that several different designs, structures, configurations, geometries and materials may be used. For example, the materials of construction may include, but are not limited to, those that are suitable for clean room processes. Suitability of these materials may depend on factors such as their degradation during use, which increases the formation of particulate matter. Bearings may also be associated with the various shaft members, axles and wheels. These bearings may also be varied to more effectively control the conveyor system. As described, each of the embodiments tend to minimize external forces acting on the carrier by limiting surfaces subject to mechanical contact. Limiting such surfaces also reduces the likelihood of particulate formation.

The invention is also directed to a method for transporting an article in a manufacturing process. The method includes providing an embodiment of the conveyor system having a carrier for retaining the article and a drive assembly for providing relative movement of carrier. The method also involves actuating the drive assembly to provide for continuous transportation of the carrier and its payload. It will be appreciated that the relative movement of the carrier is controlled by the design of a particular conveyor system.

While the present invention has been described in conjunction with a preferred embodiment, one of ordinary skill, after reading the foregoing specification, will be able to effect various changes, substitutions or equivalents, and other alterations to the compositions and methods set forth herein.

It is therefore intended that the protection granted by Letters Patent hereon be limited only by the definitions contained in the appended claims and equivalents thereof.

What is claimed is:

1. A transportation system for conveying vehicles, comprising:
a first and second rail, the first and second rails being mutually parallel from one another;
a first and second set of wheels, the first and second set of wheels respectively disposed from the first and second rails and having mutually parallel, substantially coplanar axes of rotation in movable contact with a vehicle disposed on the first and second rail; and
a third set of wheels disposed from at least one of the first and second rails, the wheels of the third set being mutually parallel and having substantially coplanar axes of rotation, and comprising:
a substantially flat outer surface and parallel raised portions, and
a substantially flat recessed portion, the recessed portion in guiding communication with the vehicle.
